(19) United States
(12) Patent Application Publication
(10) Pub. No.: US 2009/0247555 A1
(43) Pub. Date: Oct. 1, 2009

(54) 5,7-Disubstituted[1,3]Thiazolo[4,5-D]Pyrimidin-2(3H)-One Derivatives 258

(75) Inventors: Mikael Dahlstrom, Molndal (SE); Gunnar Nordvall, Sodertalje (SE); Tobias Rein, Sodertalje (SE); Ingemar Starke, Molndal (SE)

Correspondence Address: Pepper Hamilton LLP, 400 Berwyn Park, 899 Cassatt Road, Berwyn, PA, 19312-1183, US

(73) Assignee: AstraZeneca AB, Sodertalje (SE)

(21) Appl. No.: 12/411,463

(22) Filed: Mar. 26, 2009

Related U.S. Application Data
(60) Provisional application No. 61/039,463, filed on Mar. 26, 2008.

(51) Int. Cl. A61K 31/519; C07D 239/46; A61P 25/28

(52) U.S. Cl. 514/260.1; 544/255

(57) ABSTRACT

There are disclosed novel 5,7-disubstituted [1,3]thiazolo[4,5-d]pyrimidin-2(3H)-one derivatives of formula (I)

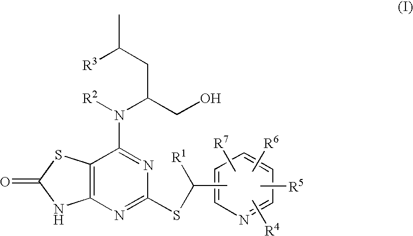

wherein R¹, R², R³, R⁴, R⁵, R⁶, and R⁷ are as defined in the specification, and pharmaceutically acceptable salts thereof, together with processes for their preparation, pharmaceutical compositions comprising them and their use in therapy. The compounds of formula (I) are CX₃CR1 receptor antagonists and are thereby particularly useful in the treatment or prophylaxis of neurodegenerative disorders, demyelinating disease, cardio- and cerebrovascular atherosclerotic disorders, peripheral artery disease, rheumatoid arthritis, is pulmonary diseases such as COPD, asthma or pain.

### FIELD OF THE INVENTION

The present invention discloses novel 5,7-disubstituted [1,3]thiazolo[4,5-d]pyrimidin-2(3H)-one derivatives together with processes for their preparation, pharmaceutical formulations comprising them and their use in therapy.

### BACKGROUND OF THE INVENTION

Chemokines play an important role in immune and inflammatory responses in various diseases and disorders, including asthma, atherosclerosis and allergic diseases, as well as autoimmune pathologies such as rheumatoid arthritis and multiple sclerosis. These small, secreted molecules are a growing superfamily of 8-14 kDa proteins characterized by a conserved cysteine motif. At the present time, the chemokine superfamily comprises four groups exhibiting characteristic structural motifs, the C—X—C, C—C and C—X₃—C and XC families. The C—X—C and C—C families have sequence similarity and are distinguished from one another on the basis of a single amino acid insertion between the NH-proximal pair of cysteine residues. The C—X₃—C family is distinguished from the other two families on the basis of having a triple amino acid insertion between the NH-proximal pair of cysteine residues. In contrast, members of the XC family lack one of the first two cysteine residues.

The C—X—C chemokines include several potent chemoattractants and activators of neutrophils such as interleukin-8 (IL-8) and neutrophil-activating peptide 2 (NAP-2).

The C—C chemokines include potent chemoattractants of monocytes, lymphocytes and neutrophils. Examples include human monocyte chemotactic proteins 1-3 (MCP-1, MCP-2 and MCP-3), RANTES (Regulated on Activation, Normal T-cell-Expressed and Secreted), eotaxin and the macrophage inflammatory proteins 1α and 1β (MIP-1α and MIP-1β).

The C—X₃—C chemokine (also known as fractalkine) is a potent chemoattractant and activator of microglia in the central nervous system (CNS) as well as of monocytes, T cells, NK cells and mast cells.

Studies have demonstrated that the actions of the chemokines are mediated by subfamilies of G protein-coupled receptors, among which are the receptors designated CCR1, CCR2, CCR2A, CCR2B, CCR3, CCR4, CCR5, CCR6, CCR7, CCR8, CCR9, CCR10 and CCR11 (for the C—C family); CXCR1, CXCR2, CXCR3, CXCR4 and CXCR5 (for the C—X—C family) and CX₃CR1 for the C—X₃—C family. These receptors represent good targets for drug development since agents that modulate these receptors would be useful in the treatment of disorders and diseases such as those mentioned above.

WO 01/25242 discloses certain thiazolo[4,5-d]pyrimidine derivatives that are useful as antagonists of receptors linked to the C—X—C and C—C chemokine families, particularly as antagonists of the CXCR2 receptor.

The present invention relates to a group of compounds that are related to compounds disclosed in WO 01/25242 but are of a structural type not specifically exemplified therein. When compared to the Examples disclosed in WO 01/25242, the compounds of the present invention display surprisingly useful properties as antagonists of the CX₃CR1 receptor.

Furthermore, compounds of the invention may have one or more of the following properties: high bioavailability, low toxicity, good pharmacokinetic properties (such as clearance), high absorption, good metabolic properties, high solubility and good solid state properties.

WO2008039138 A1 (AstraZeneca AB) discloses novel 5,7-disubstituted [1,3]thiazolo[4,5-d]pyrimidin-2(3H)one derivatives, having a pyridine ring bonded to the core system.

WO2006107257 A1 (AstraZeneca AB) discloses novel 5,7-disubstituted [1,3]thiazolo[4,5-d]pyrimidin-2(3H)one derivatives, having a phenyl ring bonded to the core system.

Walters, I. et al., Bioorganic & Medicinal Chemistry Letters (2008), 18(2), 798-803, discloses an evaluation of a series of bicyclic CXCR2 antagonists.

### BRIEF DESCRIPTION OF THE DRAWINGS

FIG. 1 is an X-ray powder diffractogram of tert-butylammonium salt 7-[(R)-1-hydroxy-4-methylpentan-2-ylamino]-5-[(1SorR)-(pyridin-2-yl)ethylthio]thiazolo[4,5-d]pyrimidin-2(3H)-one (Example 14)

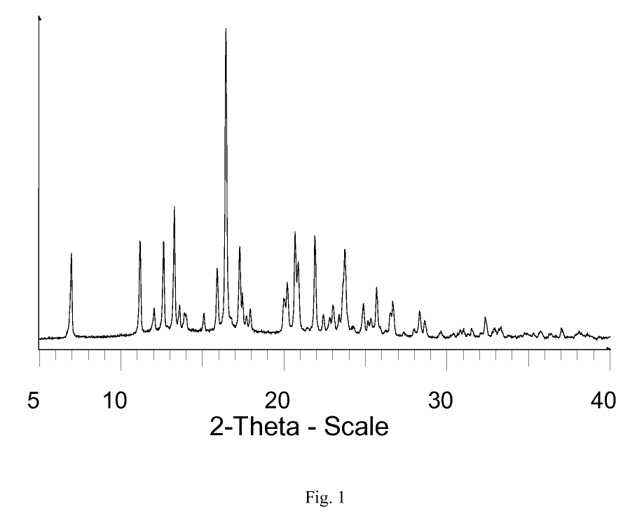

FIG. 2 is an X-ray powder diffractogram of 7-[(R)-1-hydroxy-4-methylpentan-2-ylamino]-5-[(1R)-(pyridin-2-yl)ethylthio]thiazolo[4,5-d]pyrimidin-2(3H)-one, from water slurry (Example 15).

FIG. 3 is an X-ray powder diffractogram of 7-[(R)-1-hydroxy-4-methylpentan-2-ylamino]-5-[(1R)-(pyridin-2-yl)ethylthio]thiazolo[4,5-d]pyrimidin-2(3H)-one, from ethanol slurry (Example 16).

### DISCLOSER OF THE INVENTION

The present invention provides compounds of formula (I)

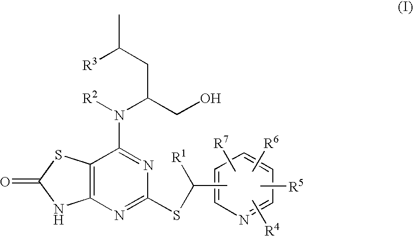

wherein:
R¹ represents CH₃ or C₂H₅;
R² represents H or CH₃;
R³ represents H or CH₃;
R⁴ represents H, CH₃, or C₂H₅;
R⁵ represents H, CH₃, or C₂H₅;
R⁶ represents H, CH₃, or C₂H₅;
R⁷ represents H, CH₃, or C₂H₅;
or a pharmaceutically acceptable salt, solvate or solvate of a salt thereof.

In another embodiment R² represents H and R³ represents CH₃.

In yet another embodiment of the invention, there is provided the compounds of Examples 1-15. In yet another embodiment, there is provided the compounds of Examples 1, 12, and 13.

The compounds of formula (I) may exist in stereoisomeric and/or tautomeric forms. It is to be understood that all enantiomers, diastereomers, racemates, tautomers and mixtures thereof are included within the scope of the invention.

When compared to the compounds disclosed in WO 01/25242, the compounds of the present invention are characterized by the presence of the branched thioalkylpyridyl at the 5-position of the thiazolopyrimidine ring system. That is, the compounds of the present invention incorporate a R¹ group that is not hydrogen.

According to the invention, there is further provided a process for the preparation of a compound of formula (I), or a pharmaceutically acceptable salt thereof which comprises reacting a compound of formula (II):

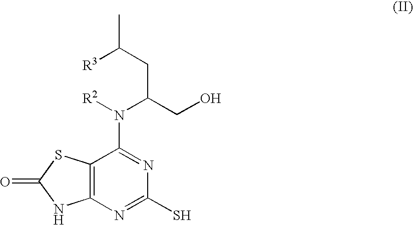

wherein R² and R³ are as defined in formula (I);
with a compound of formula (III):

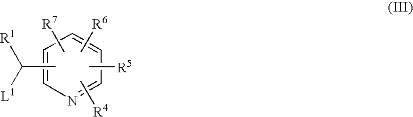

wherein R¹, R⁴, R⁵, R⁶, and R⁷ are as defined in formula (I) and L¹ represents a leaving group.

and where necessary converting the resultant compound of formula (I), or another salt thereof, into a pharmaceutically acceptable salt thereof, or converting the resultant compound of formula (I) into a further compound of formula (I); and where desired converting the resultant compound of formula (I) into an optical isomer thereof.

In the process, the reactants (II) and (III) are coupled together in a suitable organic solvent such as DMF. The reaction is optionally performed in the presence of an added organic or inorganic base such as cesium carbonate. The reaction is conducted at a suitable temperature, normally between room temperature and the boiling point of the solvent. The reaction is generally continued for a period of about one hour to one week, or until analysis indicates that formation of the required product is complete.

Suitable leaving group L¹ are halogen, particularly chloro or bromo. In one embodiment, L¹ represents chloro.

It will be apparent to a person skilled in the art that in the above processes it may be desirable or necessary to protect an amine, hydroxyl or other potentially reactive group. Suitable protecting groups and details of processes for adding and removing such groups are, in general, well known in the art. See, for example, “Protective Groups in Organic Synthesis”, 3rd Edition (1999) by Greene and Wuts.

The present invention includes compounds of formula (I) in the form of salts. Suitable salts include those formed with organic or inorganic acids or organic or inorganic bases. Such salts will normally be pharmaceutically acceptable although salts of non-pharmaceutically acceptable acids or bases may be of utility in the preparation and purification of the compound in question.

Salts of compounds of formula (I) may be formed by reacting the free compound, or a salt, enantiomer or racemate thereof, with one or more equivalents of the appropriate acid or base. The reaction may be carried out in a solvent or medium in which the salt is insoluble or in a solvent in which the salt is soluble, for example, water, dioxan, ethanol, tetrahydrofuran or diethyl ether, or a mixture of solvents, which may be removed in vacuo or by freeze drying. The reaction may also be a metathetical process or it may be carried out on an ion exchange resin.

Compounds of formula (III) are either commercially available, or known in the literature, or may be prepared using known methods that will be readily apparent to the man skilled in the art.

The compound of formula (II), wherein R² is hydrogen and R³ is methyl may be prepared as follows:

The compound having Registry No. 911715-56-5 is disclosed in WO2006107257 as Example 9.

For example, compounds of formula (I), may be prepared as shown in Scheme 1:

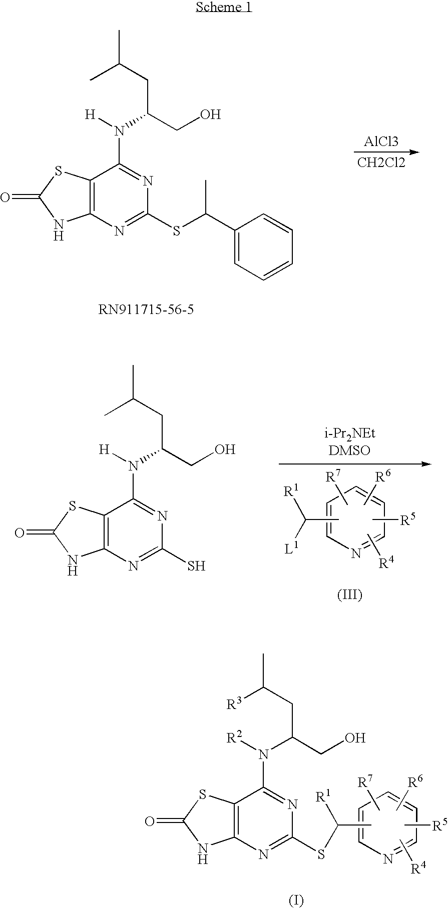

Alternatively, compounds of formula I may be prepared according to a process analogous to those described in WO2008039138 A1 and WO2006107257 A1.

Intermediate compounds may be used as such or in protected form. Suitable protecting groups and details of processes for adding and removing such groups are, in general, well known in the art. See, for example, “Protective Groups in Organic Synthesis”, 3rd Edition (1999) by Greene and Wuts.

The compounds of the invention and intermediates thereto may be isolated from their reaction mixtures and, if necessary further purified, by using standard techniques.

The compounds of formula (I) may exist in stereoisomeric forms. Therefore, all enantiomers, diastereomers, racemates and mixtures thereof are included within the scope of the invention. The various optical isomers may be isolated by separation of a stereoisomeric mixture of the compounds using conventional techniques, for example, fractional crystallisation, or HPLC. Alternatively, the various optical isomers may be prepared directly using optically active starting materials.

The compounds of formula (I) contain two stereogenic centres and may thus exist in four discrete stereoisomeric forms as shown in formulae (Ia) to (Id)

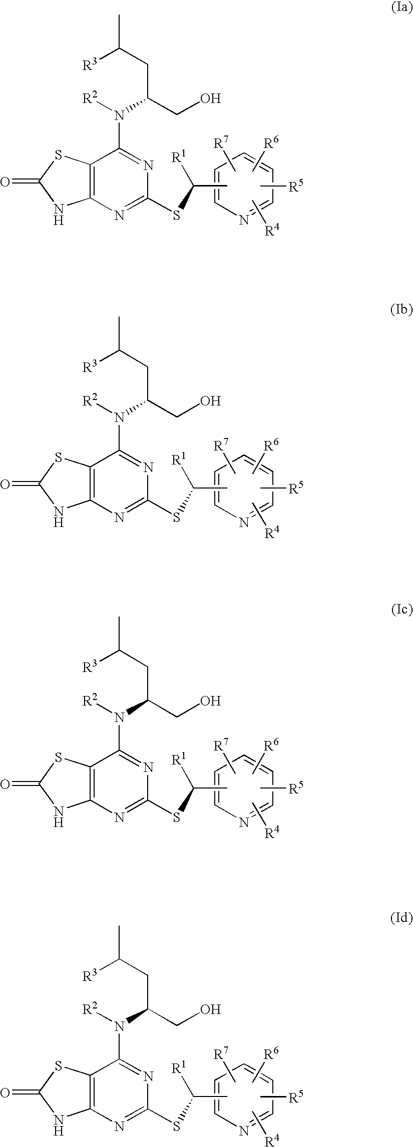

All such four stereoisomers and any mixtures thereof are included within the scope of the invention. In one embodiment, the compounds of formula (I) have the stereochemistry shown in formula (Ia). In another embodiment, the compounds of formula (I) have the stereochemistry shown in formula (Ib).

Intermediate compounds may also exist in stereoisomeric forms and may be used as purified enantiomers, diastereomers, racemates or mixtures.

In this specification the term “halo” or “halogen” refers to fluoro, chloro, bromo, and iodo. The compounds of formula (I), and their pharmaceutically acceptable salts are useful because they possess pharmacological activity as antagonists of the CX₃CR1 receptor. In particular, when compared to the compounds specifically exemplified in WO 01/25242, the compounds of formula (I) of the present invention possess significantly improved potencies for inhibition of the CX₃CR1 receptor and /or decreased potencies for inhibition of the CXCR2 receptor. Preferred compounds of the present invention display both enhanced potency for the inhibition of CX₃CR1 and decreased potency for inhibition of CXCR2.

In one aspect the present invention provides a compound of formula (I) or a pharmaceutically acceptable salt thereof, for use as a medicament.

In one aspect the present invention provides a pharmaceutical formulation comprising a compound of formula (I), or a pharmaceutically acceptable salt thereof, in admixture with a pharmaceutically acceptable diluent or carrier.

In another aspect the present invention provides the use of a compound of formula (I) or a pharmaceutically acceptable salt thereof, in the manufacture of a medicament, for the treatment or prophylaxis of diseases or conditions in which antagonism of the CX₃CR1 receptor is beneficial.

In another embodiment the present invention provides a compound of formula (I) for use in the treatment or prophylaxis of diseases or conditions in which antagonism of the CX₃CR1 receptor is beneficial.

In another aspect the present invention provides the use of a compound of formula (I) or a pharmaceutically acceptable salt thereof, in the manufacture of a medicament, for the treatment or prophylaxis of neurodegenerative disorders, demyelinating disease, cardio- and cerebrovascular atherosclerotic disorders, peripheral artery disease, rheumatoid arthritis, pulmonary diseases such as COPD, asthma or pain.

In another embodiment the present invention provides a compound of formula (I) for use in the treatment or prophylaxis of neurodegenerative disorders, demyelinating disease, cardio- and cerebrovascular atherosclerotic disorders, peripheral artery disease, rheumatoid arthritis, pulmonary diseases such as COPD, asthma or pain.

In another aspect the present invention provides the use of a compound of formula (I) or a pharmaceutically acceptable salt thereof, in the manufacture of a medicament, for the treatment or prophylaxis of multiple sclerosis (MS).

In another embodiment the present invention provides a compound of formula (I) for use in the treatment or prophylaxis of multiple sclerosis (MS).

In another aspect the present invention provides the use of a compound of formula (I) or a pharmaceutically acceptable salt thereof, in the manufacture of a medicament, for the treatment or prophylaxis of atherosclerosis by changing the composition of the plaques to reduce the risk of plaque rupture and atherothrombotic events.

In another embodiment the present invention provides a compound of formula (I) for use in the treatment or prophylaxis of atherosclerosis by changing the composition of the plaques to reduce the risk of plaque rupture and atherothrombotic events.

In another aspect the present invention provides the use of a compound of formula (I) or a pharmaceutically acceptable salt thereof, in the manufacture of a medicament, for the treatment or prophylaxis of atherosclerosis by preventing and/or reducing the formation of new atherosclerotic lesions or plaques and/or by preventing or slowing progression of existing lesions and plaques.

In another embodiment the present invention provides a compound of formula (I) for use in the treatment or prophylaxis of atherosclerosis by preventing and/or reducing the formation of new atherosclerotic lesions or plaques and/or by preventing or slowing progression of existing lesions and plaques.

In another aspect the present invention provides the use of a compound of formula (I) or a pharmaceutically acceptable salt thereof, in the manufacture of a medicament, for the treatment or prophylaxis of stroke or transient brain injury (TBI).

In another embodiment the present invention provides a compound of formula (I) for use in the treatment or prophylaxis of stroke or transient brain injury (TBI).

According to the invention, there is also provided a method of treating, or reducing the risk of, diseases or conditions in which antagonism of the CX₃CR1 receptor is beneficial which comprises administering to a person suffering from or at risk of, said disease or condition, a therapeutically effective amount of a compound of formula (I) or a pharmaceutically acceptable salt thereof.

There is also provided a method of treating, or reducing the risk of, neurodegenerative disorders, demyelinating disease, cardio- and cerebrovascular atherosclerotic disorders, peripheral artery disease, rheumatoid arthritis, pulmonary diseases such as COPD, asthma or pain in a person suffering from or at risk of, said disease or condition, wherein the method comprises administering to the person a therapeutically effective amount of a compound of formula (I) or a pharmaceutically acceptable salt thereof.

There is also provided a method of treating, or reducing the risk of, multiple sclerosis (MS) in a person suffering from or at risk of, said disease or condition, wherein the method comprises administering to the person a therapeutically effective amount of a compound of formula (I) or a pharmaceutically acceptable salt thereof.

There is also provided a method of treating, or reducing the risk of atherosclerosis by changing the composition of the plaques so as to reduce the risk of plaque rupture and atherothrombotic events in a person suffering from or at risk of, said disease or condition, wherein the method comprises administering to the person a therapeutically effective amount of a compound of formula (I) or a pharmaceutically acceptable salt thereof.

There is also provided a method of treating, or reducing the risk of atherosclerosis by preventing and/or reducing the formation of new atherosclerotic lesions or plaques and/or by preventing or slowing progression of existing lesions and plaques in a person suffering from or at risk of, said disease or condition, wherein the method comprises administering to the person a therapeutically effective amount of a compound of formula (I) or a pharmaceutically acceptable salt thereof.

There is also provided a method of treating, or reducing the risk of stroke or transient brain injury (TBI) in a person suffering from or at risk of, said disease or condition, wherein the method comprises administering to the person a therapeutically effective amount of a compound of formula (I) or a pharmaceutically acceptable salt thereof.

Another object of the present invention is the intermediate product as obtained in step a) of Example 1, ie the following compound:
7-[(R)-1-hydroxy-4-methylpentan-2-ylamino]-5-mercaptothiazolo[4,5-d]pyrimidin-2(3H)-one;
or a pharmaceutically acceptable salt, solvate or solvate of a salt thereof.

The compounds can be used as monotheraphy, or in combinations, either as prophylactic or therapeutic treatment of inflammatory conditions and diseases of the central nervous system such as stroke and transient brain injury (TBI). (Soriano et al. J. Neuroimmunology 2002, 125, 59-65.).

In another aspect the invention provides a pharmaceutical formulation comprising a therapeutically effective amount of a compound of formula (I), or a pharmaceutically acceptable salt thereof, in admixture with a pharmaceutically acceptable adjuvant, diluent or carrier, for use in the treatment or prophylaxis of diseases or conditions in which antagonism of the CX₃CR1 receptor is beneficial.

In another aspect the invention provides a pharmaceutical formulation comprising a therapeutically effective amount of a compound of formula (I), or a pharmaceutically acceptable salt thereof, in admixture with a pharmaceutically acceptable adjuvant, diluent or carrier, for use in the treatment or prophylaxis of neurodegenerative disorders, demyelinating disease, cardio- and cerebrovascular atherosclerotic disorders, peripheral artery disease, rheumatoid arthritis, COPD, asthma or pain.

In another aspect the invention provides a pharmaceutical formulation comprising a therapeutically effective amount of a compound of formula (I), or a pharmaceutically acceptable salt thereof, in admixture with a pharmaceutically acceptable adjuvant, diluent or carrier, for use in the treatment or prophylaxis of multiple sclerosis.

In another aspect the present invention provides a pharmaceutical formulation comprising a therapeutically effective amount of a compound of formula (I), or a pharmaceutically acceptable salt thereof, in admixture with a pharmaceutically acceptable adjuvant, diluent or carrier, for use in the treatment or prophylaxis of stroke or transient brain injury (TBI).

In another aspect the present invention provides a pharmaceutical formulation comprising a therapeutically effective amount of a compound of formula (I), or a pharmaceutically acceptable salt thereof, in admixture with a pharmaceutically acceptable adjuvant, diluent or carrier, for use in the treatment or prophylaxis of atherosclerosis by preventing and reducing the formation of new atherosclerotic lesions and/or plaques and/or by preventing or slowing progression of existing lesions and plaques.

The compounds of formula (I) and their pharmaceutically acceptable salts are indicated for use in the treatment or prophylaxis of diseases or conditions in which modulation of activity at the CX₃CR1 receptor is desirable. In particular, the compounds are indicated for use in the treatment of neurodegenerative disorders or demyelinating disease in mammals including man. More particularly, the compounds are indicated for use in the treatment of multiple sclerosis. The compounds are also indicated to be useful in the treatment of pain, rheumatoid arthritis, osteoarthritis, cardio- and cerebrovascular atherosclerotic disorders, peripheral artery disease and pulmonary arterial hypertension.

Conditions that may be specifically mentioned are: neurodegenerative diseases and dementia disorders, for example, Alzheimer's disease, amyotrophic lateral sclerosis and other motor neuron diseases, Creutzfeldt-Jacob's disease and other prion diseases, HIV encephalopathy, Huntington's disease, frontotemporal dementia, Lewy body dementia and vascular dementia; polyneuropathies, for example, Guillain-Barré syndrome, chronic inflammatory demyelinating polyradiculoneuropathy, multifocal motor neuropathy and plexopathies; CNS demyelination, for example, acute disseminated/haemorrhagic encephalomyelitis and subacute sclerosing panencephalitis; neuromuscular disorders, for example, myasthenia gravis and Lambert-Eaton syndrome; spinal disorders, for example, tropical spastic paraparesis and stiff-man syndrome; paraneoplastic syndromes, for example, cerebellar degeneration and encephalomyelitis; traumatic brain injury; migraine; cancer; allograft rejection; systemic sclerosis; viral infections; parasite-transmitted diseases, for example, malaria; periodontal disease; myocardial infarction; stroke; coronary heart disease; ischaemic heart disease; restenosis; rheumatoid arthritis; pulmonary diseases such as COPD; asthma or pain.

The compounds of the invention are also indicated for use in the treatment of atherosclerosis by preventing and/or reducing the formation of new atherosclerotic lesions or plaques and/or by preventing or slowing progression of existing lesions and plaques.

The compounds of the invention are also indicated for use in the treatment of atherosclerosis by changing the composition of the plaques so as to reduce the risk of plaque rupture and atherothrombotic events.

The compounds of the invention are also indicated for use in the treatment of inflammatory bowel disease (IBD), for example, Crohn's disease and ulcerative colitis, by inducing remission and/or maintaining remission of IBD.

Prophylaxis is expected to be particularly relevant to the treatment of persons who have suffered a previous episode of, or are otherwise considered to be at increased risk of, the disease or condition in question. Persons at risk of developing a particular disease or condition generally include those having a family history of the disease or condition, or those who have been identified by genetic testing or screening to be particularly susceptible to developing the disease or condition.

For the above mentioned therapeutic indications, the dosage administered will, of course, vary with the compound employed, the mode of administration and the treatment desired. However, in general, satisfactory results are obtained when the compounds are administered at a dosage of the solid form of between 1 mg and 2000 mg per day.

The compounds of formula (I) and pharmaceutically acceptable derivatives thereof, may be used on their own, or in the form of appropriate pharmaceutical compositions in which the compound or derivative is in admixture with a pharmaceutically acceptable adjuvant, diluent or carrier. Administration may be by, but is not limited to, enteral (including oral, sublingual or rectal), intranasal, intravenous, topical or other parenteral routes. Conventional procedures for the selection and preparation of suitable pharmaceutical formulations are described in, for example, “Pharmaceuticals—The Science of Dosage Form Designs”, M. E. Aulton, Churchill Livingstone, 1988. The pharmaceutical composition preferably comprises less than 80% and more preferably less than 50% of a compound of formula (I), or a pharmaceutically acceptable salt thereof.

There is also provided a process for the preparation of such a pharmaceutical composition that comprises mixing the ingredients.

The invention further relates to combination therapies wherein a compound of formula (I) or a pharmaceutically acceptable salt thereof, or a pharmaceutical composition or formulation comprising a compound of formula (I), is administered concurrently or sequentially with therapy and/or an agent for the treatment of any one of cardio- and cerebrovascular atherosclerotic disorders and peripheral artery disease.

Also claimed is a pharmaceutical composition comprising a compound of formula (I) with another therapeutic agent that is useful in the treatment of neurodegenerative disorders, demyelinating disease, cardio- and cerebrovascular atherosclerotic disorders, peripheral artery disease, rheumatoid arthritis, pulmonary diseases such as COPD, asthma, pain, multiple sclerosis, stroke or transient brain injury (TBI).

In particular, a compound of formula (I) or a pharmaceutically acceptable salt thereof may be administered in association with compounds from one or more of the following groups:
1) anti-inflammatory agents, for example,
   a) NSAIDs (e.g. acetylsalicylic acid, ibuprofen, naproxen, flurbiprofen, diclofenac, indometacin);
   b) leukotriene synthesis inhibitors (5-LO inhibitors e.g. AZD4407, Zileuton, licofelone, CJ13610, CJ13454; FLAP inhibitors e.g. BAY-Y-1015, DG-031, MK591, MK886, A81834; LTA4 hydrolase inhibitors e.g. SC56938, SC57461A);
   c) leukotriene receptor antagonists;( e.g. CP195543, amelubant, LY293111, accolate, MK571);
2) anti-hypertensive agents, for example,
   a) beta-blockers (e.g. metoprolol, atenolol, sotalol);
   b) angiotensin converting enzyme inhibitors (e.g. captopril, ramipril, quinapril, enalapril);
   c) calcium channel blockers (e.g. verapamil, diltiazem, felodipine, amlodipine);
   d) angiotensin II receptor antagonists (e.g. irbesartan, candesartan, telemisartan, losartan);
3) anti-coagulantia, for example,
   a) thrombin inhibitors (e.g. ximelagatran), heparines, factor Xa inhibitors, AZD0837;
   b) platelet aggregation inhibitors (e.g. clopidrogrel, ticlopidine, prasugel, Brilinta™);
4) modulators of lipid metabolism, for example,
   a) insulin sensitizers such as PPAR agonists (e.g. pioglitazone, rosiglitazone, Galida, muraglitazaar, gefemrozil, fenofibrate);
   b) HMG-CoA reductase inhibitors, statins (e.g. simvastatin, pravastatin, atorvastsattin, rosuvastatin, fluvastatin, pitavastatin);
   c) cholesterol absorption inhibitors (e.g. ezetimibe);
   d) IBAT inhibitors (e.g. AZD-7806);
   e) LXR agonists (e.g. GW-683965A, T-0901317);
   f) FXR receptor modulators;
   g) phospholipase inhibitors;
5) anti-anginal agents, for example, nitrates and nitrites;
6) modulators of oxidative stress, for example, anti-oxidants. (probucol), myeloperoxidase inhibitors.

The invention is illustrated, but in no way limited, by the following examples:

### General Methods

All solvents used were analytical grade and commercially available anhydrous solvents were routinely used for reactions. Reactions were typically run under an inert atmosphere of nitrogen or argon.

¹H and ¹³C NMR spectra were recorded on a Varian Mercury-300 MHz, Varian Inova-400 MHz, Varian Inova-600 MHz or on Varian Inova-500 MHz spectrometer. Peak multiplicities are shown as follows: s, singlet; m, multiplet; bs broad singlet. Unless specifically noted in the examples, spectra were recorded at 400 MHz for proton. The following reference signals were used: the middle line of DMSO-d₆ δ 2.49 (¹H); the middle line of CD₃OD δ 3.30 (¹H) and CDCl₃ δ 7.26 (¹H).

A typical workup procedure after a reaction consisted of extraction of the product with a solvent such as ethyl acetate, washing with water followed by drying of the organic phase over MgSO₄ or Na₂SO₄, and concentration of the solution in vacuo.

The following abbreviations have been used: DCM=dichloromethane; DMF=N,N-dimethylformamide.

Starting materials used were either available from commercial sources or prepared according to literature procedures and had experimental data in accordance to those reported. Hereinbelow, in Examples 12, 13 and 15, for the separation into diasteromers, the racemate formed has been reacted with a homochiral substance. Then, the product resulting therefrom has been resolved by preparative chromatography.

It should be noted that for Examples 1-11, both the (R,R) and the (R,S) isomers are envisaged.

The X-ray powder diffraction (referred to herein as XRPD) pattern was determined by mounting a sample on Bruker zero background holder, single silicon crystal, and spreading out the sample into a thin layer. Using a Bruker D8 Advance theta-2theta diffractometer with a V\dot{A}NTEC-1 detector, the sample was spun (to improve counting statistics) and irradiated with X-rays generated by a copper tube operated at 30 kV and 50 mA. Automatic variable divergence slits and secondary anti scatter slits were implemented.

The X-ray powder analysis was performed according to standard methods, which can be found in e.g. Kitaigorodsky, A. I. (1973), Molecular Crystals and Molecules, Academic Press, New York; Bunn, C. W. (1948), Chemical Crystallography, Clarendon Press, London; or Klug, H. P. & Alexander, L. E. (1974), X-ray Diffraction Procedures, John Wiley & Sons, New York.

Absolute intensities were less accurate and therefore replaced with relative intensities.

Definition relative intensities:

| | | |
|---|---|---|
| vs | Very strong | 81-100 |
| s | Strong | 41-80 |
| m | Medium | 21-40 |
| w | Weak | <20 |

It will be understood that the 2-theta values of the X-ray powder diffraction pattern may vary slightly depending on sample, instrumentation and sample preparation. It will also be understood that the relative intensities of peaks may vary according to the orientation of the sample under test so that the intensities shown in the XRPD trace included herein are illustrative and not intended to be used for absolute comparison.

### EXAMPLE 1

### 7-[(R)-1-hydroxy-4-methylpentan-2-ylamino]-5-[1-(pyridin-2-yl)ethylthio]thiazolo[4,5-d]pyrimidin-2(3H)-one

### Step a)

### 7-[(R)-1-hydroxy-4-methylpentan-2-ylamino]-5-mercaptothiazolo[4,5-d]pyrimidin-2(3H)-one;

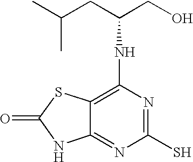

To a stirred mixture of aluminum chloride (4.0 g, 30.4 mmol) in DCM (30 ml) at room temperature was added 7-[(R)-1-hydroxy-4-methylpentan-2-ylamino]-5-[(1-phenylethyl)thio]thiazolo[4,5-d]pyrimidin-2(3H)-one (4.1 g, 10.1 mmol) in DCM (20 ml) drop wise during 20 min. The reaction mixture was stirred for 4.5 h and was then quenched with 4M NaOH to pH 12-13. The organic layer was separated, the aqueous phase was filtered and the product was precipitate out on acidification with conc. HCl. The product was filtered off and dried to give 2.7 g (89%) of the title compound.

¹H NMR (400 MHz, DMSO-d₆) δ: 0.80-0.92 (m, 6H), 1.31-1.42 (m, 2H), 1.53-1.67 (m, 1H), 3.30-3.48 (2H), 3.80 (bs, 1H), 5.10 (bs, 1H), 7.39 (bs, 1H).

### Step b)

### 7-[(R)-1-hydroxy-4-methylpentan-2-ylamino]-5-[1-(pyridin-2-yl)ethylthio]thiazolo [4,5-d]pyrimidin-2(3H)-one

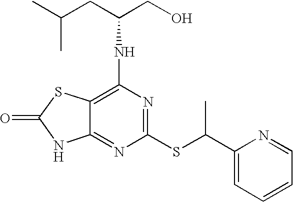

7-[(R)-1-hydroxy-4-methylpentan-2-ylamino]-5-mercaptothiazolo[4,5-d]pyrimidin-2(3H)-one (0.15 g, 0.5 mmole), 2-(1-chloroethyl)pyridine hydrochloride (0.11 g, 0.6 mmole) and cesium carbonate (0.41 g, 1.25 mmole) was mixed in DMF (2 mL) and stirred at 70° C. for 2 hours. After cooling to room temperature water and CH₂Cl₂ was added. The organic layer was separated, dried with Na₂SO₄ and the solvent was removed under reduced pressure. The residue was purified by flash chromatography on silica with CH₂Cl₂/acetone 10/3 as eluent to give 0.056 g (28%) of the title compound.

¹H NMR (400 MHz, DMSO-d₆) δ=0.78-0.90 (m, 6H), 1.30-1.48 (m, 2H), 1.50-1.70 (m, 4H), 3.22-3.44 (m, 2H), 4.13-4.36 (m, 1H), 4.98-5.08 (m, 1H), 7.19-7.29 m, 2H), 7.44-7.52 (m, 1H), 7.70-7.77 (m, 1H), 8.49-8.53 (m, 1H), 12.33 (s, 1H).

In the same manner, the following Examples 2-11 having the formula below may be prepared:

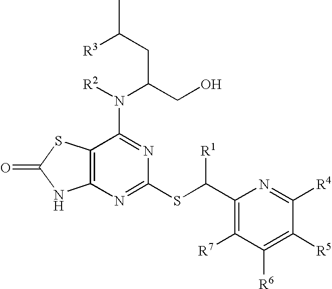

| Ex. | R¹ | R² | R³ | R⁴ | R⁵ | R⁶ | R⁷ |
|---|---|---|---|---|---|---|---|
| 2 | Methyl | H | Methyl | Methyl | H | Methyl | H |
| 3 | Methyl | H | Methyl | Methyl | H | H | H |
| 4 | Methyl | H | Methyl | H | Methyl | H | H |
| 5 | Methyl | H | Methyl | Ethyl | H | H | H |
| 6 | Methyl | H | Methyl | H | Ethyl | H | H |
| 7 | Methyl | H | Methyl | H | Methyl | H | Methyl |
| 8 | Ethyl | H | Methyl | Methyl | H | Methyl | H |
| 9 | Ethyl | H | Methyl | Methyl | H | H | H |
| 10 | Ethyl | H | Methyl | H | Methyl | H | H |
| 11 | Ethyl | H | Methyl | H | Methyl | H | Methyl |

### EXAMPLE 12

### 7-[(R)-1-hydroxy-4-methylpentan-2-ylamino]-5-[(1SorR)-(pyridin-2-yl)ethylthio]thiazolo[4,5-d]pyrimidin-2(3H)-one (isomer 1)

7-[(R)-1-hydroxy-4-methylpentan-2-ylamino]-5-[1-(pyridin-2-yl)ethylthio]thiazolo[4,5-d]pyrimidin-2(3H)-one was separated on a Chiralcel OJ: 250×50 mm, 20 μm, 40° C., 120 mL/min, with heptane/EtOH/MeOH/TEA:60/36/4/0.1 as eluent until the first peak eluated then changed to EtOH/TEA 100/0.1 then changed back after the second peak to the starting eluent. The isomer eluated first was collected and the solvent was removed under reduced pressure to give the title compound (3.32 g, 45%, 99.8% d.e.).

¹H NMR (400 MHz, DMSO-d₆) δ=0.78-0.91 (m, 6H), 1.27-1.48 (m, 2H), 1.51-1.73 (m, 4H), 3.26-3.48 (m, 2H), 4.29 (bs, 1H), 4.68 (bt, 1H), 5.05 (q, 1H), 7.17 (d, 1H), 7.21-7.28 (m, 1H), 7.48 (d, 1H), 7.72 (dt, 1H), 8.51 (bd, 1H), 12.24 (bs, 1H).

### EXAMPLE 13

### 7-[(R)-1-hydroxy-4-methylpentan-2-ylamino]-5-[(1RorS)-(pyridin-2-yl)ethylthio]thiazolo[4,5-d]pyrimidin-2(3H)-one (isomer 2)

7-[(R)-1-hydroxy-4-methylpentan-2-ylamino]-5-[1-(pyridin-2-yl)ethylthio]thiazolo[4,5-d]pyrimidin-2(3H)-one was separated on a Chiralcel OJ: 250×50 mm, 20 μm, 40° C., 120 mL/min, with Heptane/EtOH/MeOH/TEA:60/36/4/0.1 as eluent until the first peak eluated then changed to EtOH/TEA 100/0.1 then changed back after the second peak to the starting eluent. The second peak was collected and the solvent was removed under reduced pressure to give the title compound (3.92 g, 54%, 99.9% d.e.)

¹H NMR (400 MHz, DMSO-d₆) δ=0.80 (d, 3H), 0.86 (d, 3H), 1.30-1.48 (m, 2H), 1.50-1.63 (m, 1H), 1.67 (d, 3H), 3.28-3.48 (m, 2H), 4.12-4.26 (m, 1H), 4.67 (bt, 1H), 5.02 (q, 1H), 7.11 (bs, 1H), 7.22-7.27 (m, 1H), 7.46 (d, 1H), 7.72 (dt, 1H), 8.49-8.52 (m,1H), 12.32 (bs, 1H).

### EXAMPLE 14

### 7-[(R)-1-hydroxy-4-methylpentan-2-ylamino]-5-[(1SorR)-(pyridin-2-yl)ethylthio]thiazolo[4,5-d]pyrimidin-2(3H)-one, tert-butylammonium salt

The 7-[(R)-1-hydroxy-4-methylpentan-2-ylamino]-5-[(1SorR)-(pyridin-2-yl)ethylthio]thiazolo[4,5-d]pyrimidin-2(3H)-one (0.558 g, 1.38 mmol) was dissolved in ethyl acetate (2.5 mL). tert-Butylamine (0.151 g, 2.06 mmol) was added dropwise during a couple of min. A white solid precipitated almost instantaneously and after stirring the mixture overnight the material was filtered off. Drying at 45° C. under reduced pressure overnight yielded the tert-butylamine salt of the 7-[(R)-1-hydroxy-4-methylpentan-2-ylamino]-5-[(1SorR)-(pyridin-2-yl)ethylthio]thiazolo[4,5-d]pyrimidin-2(3H)-one (0.521 g, 1.09 mmol, 78%) as a white solid.

¹H NMR (400 MHz, DMSO-d₆) δ 0.83 (d, 3H), 0.86 (d, 3H), 1.23 (s, 8H), 1.29-1.49 (m, 2H), 1.50-1.62 (m, 1H), 1.64 (d, 3H), 3.26 (dd, 1H), 3.38 (dd, 1H), 4.09-4.33 (m, 1H), 5.08 (q, 1H), 5.78 (d, 1H), 7.22 (dd, 1H), 7.45 (d, 1H), 7.70 (dd, 1H), 8.49 (d, 1H).

TABLE 1
XRPD of the product from Example 14

| Angle | d value | Intensity % |
|---|---|---|
| 2-Theta ° | Angstrom | % |
| 6.9 | 12.7 | m |
| 11.2 | 7.9 | m |
| 12.0 | 7.4 | w |
| 12.6 | 7.0 | m |
| 13.3 | 6.7 | s |
| 13.6 | 6.5 | w |
| 13.9 | 6.4 | w |
| 15.1 | 5.9 | w |
| 15.9 | 5.6 | m |
| 16.4 | 5.4 | vs |
| 17.3 | 5.13 | m |
| 17.4 | 5.08 | w |
| 17.7 | 5.01 | w |
| 17.9 | 4.94 | w |
| 20.0 | 4.43 | w |
| 20.2 | 4.39 | w |
| 20.7 | 4.29 | m |
| 20.9 | 4.25 | m |
| 21.9 | 4.05 | m |
| 22.4 | 3.96 | w |
| 22.8 | 3.89 | w |
| 23.0 | 3.86 | w |
| 23.4 | 3.80 | w |
| 23.8 | 3.74 | m |
| 24.9 | 3.57 | w |
| 25.2 | 3.53 | w |
| 25.4 | 3.51 | w |
| 25.7 | 3.46 | w |
| 26.6 | 3.35 | w |
| 26.7 | 3.34 | w |
| 28.4 | 3.15 | w |
| 28.7 | 3.11 | w |
| 32.4 | 2.76 | w |

### EXAMPLE 15

### 7-[(R)-1-hydroxy-4-methylpentan-2-ylamino]-5-[(1R)-(pyridin-2-yl)ethylthio]thiazolo[4,5-d]pyrimidin-2(3H)-one, hydrate

7-[(R)-1-hydroxy-4-methylpentan-2-ylamino]-5-[(1SorR)-(pyridin-2-yl)ethylthio]thiazolo[4,5-d]pyrimidin-2(3H)-one, tert-butanol solvate (50 mg) was slurried in approximately 1.5 ml water for 10 days with a magnetic stirrer, which afforded crystalline 7-[(R)-1-hydroxy-4-methylpentan-2-ylamino]-5-[(1SorR)-(pyridin-2-yl)ethylthio]thiazolo[4,5-d]pyrimidin-2(3H)-one, hydrate.

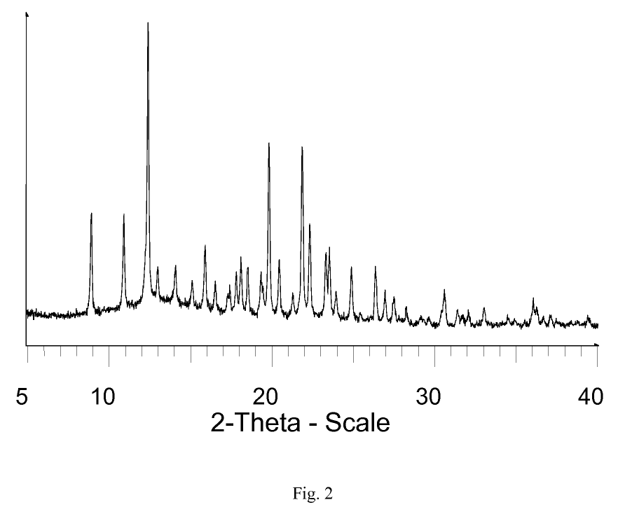

In an upscaled version, to 600 mg of crystalline 7-[(R)-1-hydroxy-4-methylpentan-2-ylamino]-5-[(1SorR)-(pyridin-2-yl)ethylthio]thiazolo[4,5-d]pyrimidin-2(3H)-one, tert-butanol solvate in a vial, approximatly 10 ml of water was added. The slurry was stirred on a magnetic stirrer for 3 days. The slurry was left on a petri dish to dry for at least 24 hours. This afforded crystalline 7-[(R)-1-hydroxy-4-methylpentan-2-ylamino]-5-[(1SorR)-(pyridin-2-yl)ethylthio]thiazolo[4,5-d]pyrimidin-2(3H)-one, hydrate according to FIG. 2.

TABLE 2
XRPD of the product from Example 15

| Angle | d value | Intensity % |
|---|---|---|
| 2-Theta ° | Angstrom | % |
| 8.9 | 10.0 | m |
| 10.9 | 8.1 | m |
| 12.4 | 7.2 | vs |
| 13.0 | 6.8 | w |
| 14.1 | 6.3 | w |
| 15.1 | 5.9 | w |
| 15.9 | 5.6 | m |
| 16.5 | 5.4 | w |
| 17.4 | 5.1 | w |
| 17.8 | 4.98 | w |
| 18.1 | 4.90 | m |
| 18.5 | 4.79 | w |
| 19.3 | 4.59 | w |
| 19.8 | 4.48 | s |
| 20.4 | 4.34 | m |
| 21.8 | 4.06 | s |
| 22.3 | 3.98 | m |
| 23.3 | 3.81 | m |
| 23.5 | 3.78 | m |
| 23.9 | 3.72 | w |
| 24.9 | 3.58 | w |
| 26.4 | 3.38 | w |
| 26.9 | 3.31 | w |
| 30.6 | 2.92 | w |

The hydrate may be described by its strongest peaks in the XRPD at d value 7.2, 4.48, 4.06 or, by including more peaks: 10.0, 8.1, 7.2, 5.6, 4.90, 4.48, 4.06, 3.98, 3.81, 3.78.

### EXAMPLE 16

### 7-[(R)-1-hydroxy-4-methylpentan-2-ylamino]-5-[(1SorR)-(pyridin-2-yl)ethylthio]thiazolo[4,5-d]pyrimidin-2(3H)-one, ethanol solvate

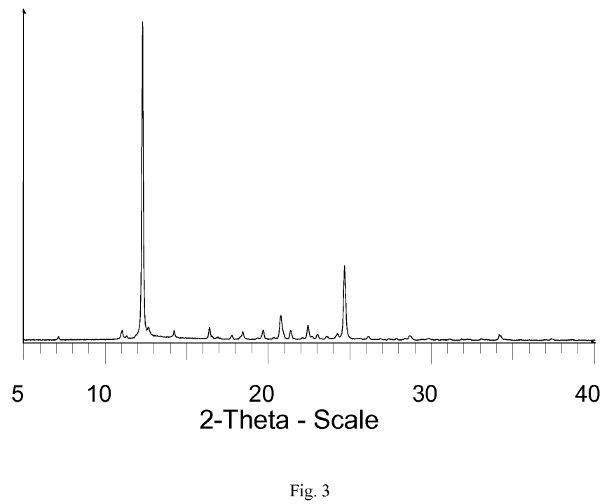

45 mg 7-[(R)-1-hydroxy-4-methylpentan-2-ylamino]-5-[(1SorR)-(pyridin-2-yl)ethylthio]thiazolo[4,5-d]pyrimidin-2(3H)-one (isomer 1) (amorphous), was dissolved in 0.5 ml 60% ethanol, by heating to about 60 C. The mixture was left overnight at room temperature, affording crystalline 7-[(R)-1-hydroxy-4-methylpentan-2-ylamino]-5-[(1SorR)-(pyridin-2-yl)ethylthio]thiazolo[4,5-d]pyrimidin-2(3H)-one, ethanol solvate according to FIG. 3.

TABLE 3
XRPD of the product from Example 16

| Angle | d value | Intensity % |
|---|---|---|
| 2-Theta ° | Angstrom | % |
| 11.0 | 8.0 | w |
| 12.3 | 7.2 | vs |
| 12.6 | 7.0 | w |
| 14.2 | 6.2 | w |
| 16.4 | 5.4 | w |
| 18.4 | 4.81 | w |
| 19.7 | 4.51 | w |
| 20.8 | 4.27 | w |
| 21.4 | 4.15 | w |
| 22.4 | 3.96 | w |
| 24.7 | 3.60 | m |

The ethanolate may be described by its strongest peak in the XRPD at d value 7.2 or, by including more peaks: 7.2, 3.60

### Fractalkine (CX3CR1) GTPgS Inhibition Assay (96 Well)

### Introduction

The activation of the CX3CR1 receptor by fractalkine can be detected using a GTPγS assay. ³⁵S-labelled GTPγS is added to the reaction (together with compounds and CX3CR1 CHO K1 membranes) to bind to the G-protein in the membranes instead of GTP upon activation of the receptor. Since ³⁵S-labelled GTPγS is non-hydrolysable the receptor will stay in its active state. The bound ³⁵S-labelled GTPγS is separated from free by vacuum filtration onto 96 well filters. In inhibition assay mode the receptor in the membranes will be activated with the EC₈₀ of fractalkine and further the CX3CR1 antagonists will inhibit the signal generated by the EC₈₀ of fractalkine.

### Reagents

Ligand: Fractalkine, PeproTech, cat no 300-31, 1 mg, −20° C. Diluted in dH₂O+0.05% BSA to a stock concentration of 0.1 mM, Stored in −20° C.

³⁵S-GTPγS, Perkin Elmer (NEN), Cat no NEG030H, Specific activity 1250 Ci/mmol, 1 mCi/ml, −20° C.

Membranes: membranes are prepared inhouse from CX3CR1 CHO K1 cells purchased from Euroscreen. Protein concentration: 5.7 mg/ml, −80° C.

### Reagents:

### 2×Assay Buffer:

HEPES: Sigma H-3375, RT
MgCl₂×6H₂O: Merck, cat no 5833, RT
NaCl: Merck, cat no 6404, RT
GDP: −20° C., Sigma cat no G-7127, Diluted in dH₂O to a stock concentration of 10 mM.
Stored in −20° C.
Gelatine type A: Sigma, cat no G2625, 1% stock in water (heating is necessary to dissolve gelatine) made fresh on the day of the experiment RT

### Wash Buffer:

Tris: Tris-HCl: Sigma, cat no T1503, RT
   Tris-base: Sigma, cat no T3283, RT
MgCl₂×6H₂O: Merck, cat no 5833, RT
NaCl: Merck, cat no 6404, RT
Assay plates: 96 well, Nunc, 269620
Filter: Printed Filtermat B, Wallac
Scintillator sheets: MeltiLex B, PerkinElmer

### Method

Assay plates are prepared by CMT with 1 compound/plate. 2 μl compound with the highest concentration 3.3 mM diluted with a factor 3 in 10 steps. This gives a final assay concentration of 33 μM for the highest concentration.

100 μl membranes (5 μg/well) diluted in 2× assay buffer is added to the assay plate.

| 2x assay buffer: | Final assay concentration |
|---|---|
| 100 mM HEPES, pH 7.4 | 50 mM |
| 200 mM NaCl | 100 mM |
| 10 mM MgCl₂ × 6H₂O | 5 mM |
| 20 μM GDP | 10 μM |
| 0.02% Gelatine | 0.01% Gelatine |

100 μl ³⁵S-GTPγS (final assay concentration: 0.00056 μCi/μl) diluted in dH₂O with EC₈₀ of fractalkine is then added. To the control wells for 100% inhibition ³⁵S-GTPγS (final assay concentration: 0.00056 μCi/μl) diluted in dH₂O is added without the EC₈₀ of fractalkine.

The reagents in the assay plate are mixed and the plate incubated in 30° C. for 1 hour.

The plate is filtrated using a Micro96 harvester and the filter is incubated in 50° C. for 1 hour.

The filter is covered with a melt-on scintillator sheet (MeltiLex), which is melted on using a MeltiLex heatsealer.

The plate is read in a microbeta.

The compounds and control are added to the dilution plate according to this pattern

| | | | | | | | | | | | |
|---|---|---|---|---|---|---|---|---|---|---|---|
| 1 | 2 | 3 | 4 | 5 | 6 | 7 | 8 | 9 | 10 | 11 | 12 |

A
B
C
D
E
F
G
H

A1, B1, C1, A12, B12, C12-0% effect, EC₈₀ of fractalkine, 2 μl DMSO
D1, E1, F1, D12, E12, F12-100% effect, no EC₈₀ of fractalkine, 2 pl DMSO
A2-11—Example 1
G1, H2, G12, H12—empty

Each substance dilution series is run on three plates, ie in triplicate.

### Calculation Method

The molar concentration (IC₅₀) of test compound, producing 50% inhibition of agonist stimulation, was derived using a program to fit data by a Sigmoidal Dose-Response Model.

### Equation

fit=(A+((B−A)/(1+((C/×)\hat{0}D)))) wherein
A—curve bottom
B—curve top
C—EC₅₀
D—slope (Hill coefficient)
X—concentration of test compound

Hereinbelow is a table showing IC₅₀ (μM) for Examples 1, 12, and 13. Compounds of the invention having a IC₅₀ lower than 10 μM are considered as being active.

| Example | IC₅₀ (μM) |
|---|---|
| 1 | 2.6 |
| 12 | 1.0 |
| 13 | 9.2 |

The compounds of the present invention wherein R¹ represents Me (containing a branched thioalkylpyridyl group in 5-position) are both more potent antagonists at the CX₃CR1 receptor and/or less potent antagonists at the CXCR2 receptor than corresponding reference compounds wherein R¹ represents H. Such enhanced selectivity with respect to antagonism of the CX₃CR1 receptor may result in significant therapeutic benefit.

### Human Whole Blood Blow Adhesion Assay

Flow adhesion assays were performed using a microfluidics system (Cellix Microfluidics SP1.0 platform) and 8-channel biochips (Cellix Vena 8 biochips). Channel 1 was used as control and channels 2 to 8 to evaluate seven different concentrations of antagonist. To 25 monitor adhesion to fractalkine, channels were first coated with 15 μl of anti-His Tag antibody at 25 μg/ml for 2 hours. Channels were then washed three times with 15 μl DPBS to remove unbound antibody. Subsequently, 15 μl of recombinant human fractalkine at 2.5 μg/ml was added to each channel and biochips were left at 4° C. overnight in a humidified atmosphere. The following day, channels were washed three times with 15 μl DPBS prior to blocking with 15 μl of BSA at 10 mg/ml for 30 minutes. Human whole blood was obtained by venipuncture using heparin as anti-coagulant. DMSO carrier or compounds in DMSO were added to 0.3 ml blood in 1.5 ml Eppendorf tubes followed by incubation at room temperature for 15 minutes. The Vena 8 biochip was placed on a microscope stage and channels were washed with RPMI 1640 medium containing 1% BSA in preparation for the assays. 6 μl blood was removed from each Eppendorf tube and used to flow through a single channel of the Vena 8 biochip using a shear stress of 2.25 dynes/cm² and whole blood geometry settings in the FlowAssay software, and using RPMI 1640 containing 1% BSA as buffer in the system. After flow for 10 minutes, the shear stress was increased to 2.25 dynes/cm² for 2 minutes before capturing 4 images of each channel at defined locations. Adhered cells were subsequently quantified using DucoCell software (Cellix).

Example 12 showed an IC50 value of 2.4 μM in the human whole blood flow adhesion assay.

1. A compound of formula (I) 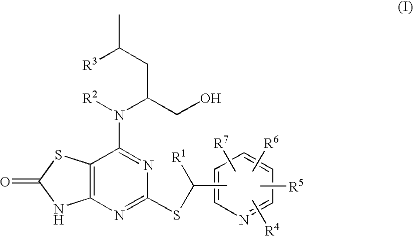
wherein:
R¹ represents CH₃ or C₂H₅;
R² represents H or CH₃;
R³ represents H or CH₃;
R⁴ represents H, CH₃, or C₂H₅;
R⁵ represents H, CH₃, or C₂H₅;
R⁶ represents H, CH₃, or C₂H₅;
R⁷ represents H, CH₃, or C₂H₅;
or a pharmaceutically acceptable salt, solvate or solvate of a salt thereof.

2. A compound of claim 1, wherein R² represents H and R³ represents CH₃.

3. A compound selected from:
7-[(R)-1-hydroxy-4-methylpentan-2-ylamino]-5-[1-(pyridin-2-yl)ethylthio]thiazolo[4,5-d]pyrimidin-2(3H)-one;
7-[(R)-1-hydroxy-4-methylpentan-2-ylamino]-5-[1-(2,4-dimethylpyridin-2-yl)ethylthio]thiazolo[4,5-d]pyrimidin-2(3H)-one;
7-[(R)-1-hydroxy-4-methylpentan-2-ylamino]-5-[1-(2-methylpyridin-2-yl)ethylthio]thiazolo[4,5-d]pyrimidin-2(3H)-one;
7-[(R)-1-hydroxy-4-methylpentan-2-ylamino]-5-[1-(3-methylpyridin-2-yl)ethylthio]thiazolo[4,5-d]pyrimidin-2(3H)-one;
7-[(R)-1-hydroxy-4-methylpentan-2-ylamino]-5-[1-(2-ethylpyridin-2-yl)ethylthio]thiazolo[4,5-d]pyrimidin-2(3H)-one;
7-[(R)-1-hydroxy-4-methylpentan-2-ylamino]-5-[1-(3-ethylpyridin-2-yl)ethylthio]thiazolo[4,5-d]pyrimidin-2(3H)-one;
7-[(R)-1-hydroxy-4-methylpentan-2-ylamino]-5-[1-(3,5-dimethylpyridin-2-yl)ethylthio]thiazolo[4,5-d]pyrimidin-2(3H)-one;
7-[(R)-1-hydroxy-4-methylpentan-2-ylamino]-5-[1-(2,4-dimethylpyridin-2-yl)propylthio]thiazolo[4,5-d]pyrimidin-2(3H)-one;
7-[(R)-1-hydroxy-4-methylpentan-2-ylamino]-5-[1-(2-methylpyridin-2-yl)propylthio]thiazolo[4,5-d]pyrimidin-2(3H)-one;
7-[(R)-1-hydroxy-4-methylpentan-2-ylamino]-5-[1-(3-methylpyridin-2-yl)propylthio]thiazolo[4,5-d]pyrimidin-2(3H)-one;
7-[(R)-1-hydroxy-4-methylpentan-2-ylamino]-5 -[1-(3,5-dimethylpyridin-2-yl)propylthio]thiazolo[4,5-d]pyrimidin-2(3H)-one;
7-[(R)-1-hydroxy-4-methylpentan-2-ylamino]-5-[(1SorR)-(pyridin-2-yl)ethylthio]thiazolo[4,5-d]pyrimidin-2(3H)-one;
7-[(R)-1-hydroxy-4-methylpentan-2-ylamino]-5-[(1RorS)-(pyridin-2-yl)ethylthio]thiazolo[4,5-d]pyrimidin-2(3H)-one,
or a pharmaceutically acceptable salt, solvate or solvate of a salt thereof.

4. (canceled)

5. A pharmaceutical formulation comprising a compound according to claim 1, or a pharmaceutically acceptable salt thereof, in admixture with a pharmaceutically acceptable diluent or carrier.

6-10. (canceled)

11. A method of treating, or reducing the risk of neurodegenerative disorder, demyelinating disease, cardio- and cerebrovascular atherosclerotic disorder, peripheral artery disease, rheumatoid arthritis, pulmonary disease, or pain, which comprises administering to a person suffering from or susceptible to such a disease or condition, a therapeutically effective amount of a compound according to claim 1, or a pharmaceutically acceptable salt thereof.

12. A method of treating, or reducing the risk of multiple sclerosis, which comprises administering to a person suffering from or susceptible to multiple sclerosis, a therapeutically effective amount of a compound according to claim 1, or a pharmaceutically acceptable salt thereof.

13. A method of treating or prophylaxis of atherosclerosis by changing the composition of the plaques to reduce the risk of plaque rupture and atherothrombotic event, which comprises administering to a person suffering from or susceptible to atherosclerosis, a therapeutically effective amount of a compound according to claim 1, or a pharmaceutically acceptable salt thereof.

14. A pharmaceutical composition comprising a compound according to claim 1 and another therapeutic agent that is useful in the treatment of neurodegenerative disorder, demyelinating disease, cardio- and cerebrovascular atherosclerotic disorder, peripheral artery disease, rheumatoid arthritis, pulmonary disease, pain, multiple sclerosis, stroke or transient brain injury (TBI).

15. 7-[(R)-1-hydroxy-4-methylpentan-2-ylamino]-5-mercaptothiazolo[4,5-d]pyrimidin-2(3H)-one;
or a pharmaceutically acceptable salt, solvate or solvate of a salt thereof.

16. A method of claim 11 wherein the pulmonary disease is chronic obstructive pulmonary disease or asthma.

17. A method treating or prophylaxis of stroke or transient brain injury (TBI), which comprises administering to a person suffering from or susceptible to stroke or TBI, a therapeutically effective amount of a compound according to claim 1, or a pharmaceutically acceptable salt thereof.
